(19) United States
(12) Patent Application Publication
(10) Pub. No.: US 2004/0210218 A1
(43) Pub. Date: Oct. 21, 2004

(54) Vertebral implant for bone fixation or interbody use

(76) Inventors: Robert A. Dixon, Powell, OH (US); Donald J. Hackman, Columbus, OH (US)

Correspondence Address: HAHN LOESER & PARKS, LLP, TWIN OAKS ESTATE, 1225 W. MARKET STREET, AKRON, OH, 44313, US

(21) Appl. No.: 10/419,011

(22) Filed: Apr. 18, 2003

(51) Int. Cl. A61B 17/56

(52) U.S. Cl. 606/69

(57) ABSTRACT

The present invention provides a biodegradable implant which can be used as fixation and/or interbody implants. The implant is formed of a biodegradable material and may be used as a cervical stabilizing system. The stabilizing system comprises a body constructed of a biodegradable, polymeric material, which when implanted within the body will maintain a predetermined structural integrity for at least a predetermined period of time while minimizing reactivity with adjacent tissues. In an embodiment of the invention, the stabilization system comprises a fixation member which includes apertures to allow selective coupling to bone segments by means of biodegradable screws. In another embodiment, the stabilization system includes a bone column implant which maintains space between at least two bone segments of a bone column. The body member is dimensioned to substantially maintain the distance, geometry and continuity between the at least two bone segments. The invention is also directed to a combination device comprising a fixation device along with an interbody implant and methods for using the stabilization system.

### FIELD OF THE INVENTION

The invention relates generally to implantable medical devices and their methods of use for stabilizing bone and/or bone attachments. More particularly, the invention is directed to implantable medical devices fabricated of biodegradable material and their methods of use.

### BACKGROUND OF THE INVENTION

Bone healing requires approximation and stabilization of the adjoining bone segments. If motion occurs across the bone segment surfaces, or if a gap is allowed to develop between two bone segments, normal bone healing is impaired. This is due to the inhibition of both the nutrient vessel ingrowth and the cellular bridging which can occur across small gaps between the adjoining bone segments. Implants can be used to stabilize an area of bone healing by allowing appropriate growth and repair. Implants can also provide a conduit for bone healing while helping to maintain the appropriate bone alignment. Various cervical column problems are resolved by the use of grafts, which will heal or “fuse” with the vertebrae. The use of spinal implants to facilitate realignment and/or fixation of spinal elements provides an effective approach to treatments for cervical column disorders. Such implants have included plates, which are bent to conform to the vertebrae, and along the spinal axis to maintain lordosis. Such plates are attached to the vertebrae using screws in different configurations. Bicortical screw purchase (where the screw penetrates the near side and the far side of the vertebrae) has been favored because of the increased strength of the construct and increased screw thread area within the bone. These screws are more technically challenging to place and implanting them adds an increased risk of morbidity from neural canal penetration. The reduced strength and decreased thread area of a unicortical screw purchase (where the screw penetrates only the near side of the vertebra) increases the probability of screw backout or loosening which may result in esophageal or other injury. Screw backout and loosening has led to the development of mechanisms for locking the screw head to the plate in unicortical screw plate designs. Such locking mechanisms not only prevent screw backout, they also reduce the tendency of the screw head to pivot within the plate. These devices contain many intricate components that increase the cost and reduce reliability of stabilizer systems. The unicortical metal devices presently available are also relatively rigid devices.

Alternatively, an interbody implant is most commonly used to replace a void from a resected disk or bone segment within a column of bones. The bone column alignment is to be curved in lordosis or kyphosis. Lordosis, kyphosis and the need for bone column alignment is also well known to those practiced in the art.

As an example, surgical interbody spinal fusion techniques allow bridging of bone tissue in continuity between adjacent vertebral bodies and across the disk space to substantially eliminate relative motion between the adjacent vertebral bodies. An implant to facilitate fusion is used as a temporary bridge between adjacent vertebral bodies.

Once surgical exposure is achieved and bone column alignment is obtained, the surgeons' usual choices for interbody implants are of bone grafts. Use of local bone would encourage bone healing, but would not prevent subsidence and loss of lordotic sagittal alignment. Use of local bone is limited due to the non-weight bearing capabilities of the pieces obtained during resection of bony outgrowths or portions of the bone. Structural support between the bone segments allows replacement of a surgical void and continuity between adjacent bone segments of the bone column. Development of bone morphogenic proteins (BMPs) as graft substitutes also provides an opportunity to avoid host site complications from bone graft harvesting, and allograft complications such as prion transfer, rejection and increased pseudo-arthrosis rate. Implanting metallic or other non-absorbable interbody devices adds risk due to the persistence of these devices after bone healing has occurred. Implanted metallic devices do not adapt to normal bony changes that occur with aging and healing/repair. Metallic implants tend to stress shield the healing bone. Breakage of a metallic implant, most commonly through repetitive stress, can result in sudden load transfer to the previously stress shielded bone. This sudden transfer of load can result in latent fracture of the healed bone segments.

Latent infection can occur in the implant site if bacteremia occurs. Infections involving metallic implants are much more difficult to treat and may require surgical removal of the implant. Breakage or migration of the implant may cause further damage to local tissues and may also require surgical removal of the implant.

Bioabsorbable (also known as biodegradable and bioresorbable) materials will absorb and may allow complete bone ingrowth versus non-resorbable implants.

Nonmetal implants are preferred also because of the minimal interference with X-rays and magnetic resonant imaging (MRI) techniques used for postoperative evaluation. Absorption of the implant over time will eliminate possible internal injury, a second operation, re-fracture, as well as magnetic, ad radiographic artifact.

Polymeric implants have been developed, however none have been successful as bone calcium implants because of material failure or tissue reactivity. Present polymeric stabilization implants do not provide the desired structural integrity, such as provided by metal bone fixation plates. The limitations of polymeric material properties have prevented effective use of such materials in bone stabilization procedures. The strongest biocompatible polymers available have a tensile strength 1/25 that of titanium and they are 50 times more elastic than titanium. Such characteristics make it difficult to operate with a minimum of stress concentrations and have the highest possible fatigue endurance limit.

### SUMMARY OF THE INVENTION

Based upon the foregoing, the present invention provides a biodegradable implant that overcomes the limitations of the prior art and achieves desired results for use as fixation implants and/or interbody implants. The invention is directed to a biodegradable cervical stabilizing system comprising a body constructed of a biodegradable, polymeric material, which when implanted within the body will maintain a predetermined structural integrity for at least a predetermined period of time while minimizing reactivity with adjacent tissues. In an embodiment of the invention, the stabilization system comprises a fixation member that includes apertures to allow selective coupling to bone segments by means of biodegradable screws. In another embodiment, the stabilization system includes a bone column implant that maintains space between at least two bone segments of a bone column. The implant includes a posterior side positionable toward the posterior portion of a bone column, and having an axis which substantially passes through the central portion of the at least two bone segments. The body member is dimensioned to substantially maintain the distance, geometry and continuity between the at least two bone segments. The invention is also directed to a combination device comprising a fixation device along with an interbody implant and methods for using the stabilization system according to the invention are also set forth.

### BRIEF DESCRIPTION OF THE DRAWINGS

FIG. 1 is a sectional view showing an embodiment of a stabilization system according to the invention, implanted on the cervical portion of a human spinal column.

FIGS. 2 and 3 show top and bottom perspective views of an alternative embodiment of the stabilizing system according to the invention.

FIG. 4 is a picture showing a cross-sectional segment of a vertebral column from an animal showing a portion of the stabilizing system according to the embodiment as shown in FIGS. 2 and 3.

FIG. 5 shows an embodiment of an interbody implant according to the invention.

FIG. 6 shows an embodiment wherein a fixation plate is selectively attached to an interbody implant according to the invention.

FIG. 7 shows a stabilization system according to the invention with an interbody implant having a fixation plate integral therewith.

FIG. 8 is a sectional view showing the embodiment of FIG. 7 implanted on the cervical portion of a human spinal column.

### DESCRIPTION OF INVENTION EMBODIMENTS

For simplification, the stabilizer system according to the invention will be described as a cervical stabilizer and embodiments thereof. Other embodiments are contemplated, wherein the components of the stabilizing system can be configured for the particular application. As the invention is directed to biodegradable stabilizing systems for use within the body, the invention can be adapted for other uses beyond cervical or bone column stabilization. For example, the device may function as a bone cement restrictor or for stabilization of bones in other areas of the body.

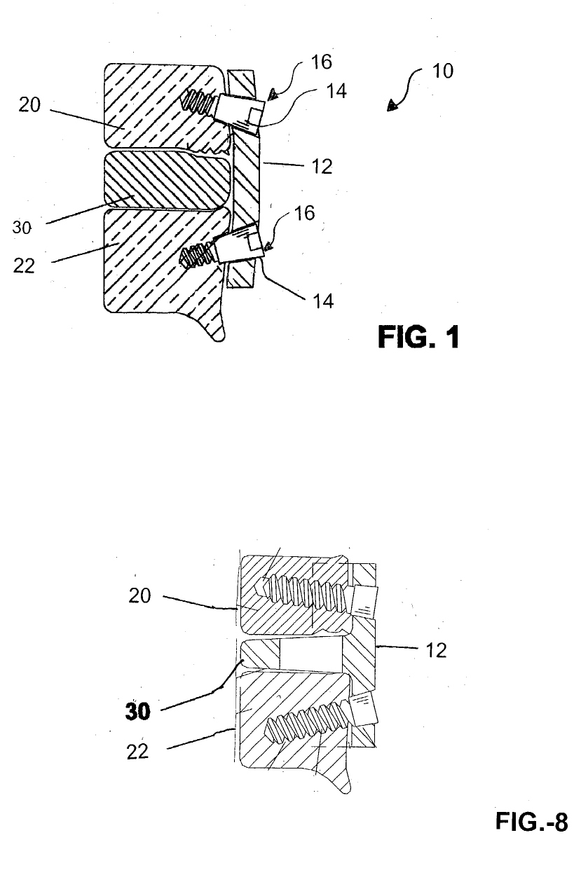

Referring to FIG. 1, a stabilization system 10 according to the invention comprises a stabilization plate 12 having a plurality of apertures 14. The stabilization plate 12 is retained in conjunction with at least two bone segments, such as the between two vertebrae 20 and 22, by means of screws 16. Certain characteristics of the stabilization plate 12 and screws 16 may be similar to that as described in co-pending U.S. patent application Ser. No. 10/083,332, filed Feb. 25, 2002, U.S. patent application Ser. No. 10/034,815, filed Dec. 27, 2001, U.S. patent application Ser. No. 09/996,858, filed Nov. 20, 2001 and U.S. patent application Ser. No. 09/977,663, filed Oct. 15, 2001, all of which are hereby incorporated by reference herein. Particularly, the screws 16 may have a locking taper, which improves screw strength and lock the screw within the bone by extending a tapered unthreaded section of the screw shank into the bone. In conjunction with use of the stabilization plate 12, a graft 30 may be used between the two vertebrae 20 and 22. The graft 30 may be a non-degrading bone growth-compatible material as an example. The stabilization plate 12, and the surrounding ligaments, tendons and muscles, are desirably preloaded to maintain compression between the graft 30 and the adjacent vertebra during motion of the body.

In the embodiment as shown, it is desirable to form plate 12 as well as screws 16 of a biodegradable material, but also a material which has both structural characteristics as well as degradation rates which are compatible for use as cervical bone column structural implants. Further, the device 10 should be substantially non-reactive with adjacent tissues. In this regard, the stabilization plate 12 may be formed of a caprolactone monomer and screws 16 may be formed of other biodegradable polymers. Caprolactone monomer is a hexagonal carbon ring with one carbon atom replaced by an oxygen atom, and a lactone attached as a side chain off of one of the other carbon rings. The oxygen atoms and the lactone bond serve as electron sources for chemical polymerization bonding. Monomers of this lactone are well described in the literature. In the present invention the commercially available epsilon configuration, in which the lactone is positioned adjacent to the chain/ring oxygen atom, has been found to provide desirable characteristics. Caprolactone materials are degraded in animal tissue by hydrolysis. Animal studies with caprolactone implants according to the invention (Sprague Dawley rats) at 3 and 6 months have shown the tissue reactivity to consist of fibroblasts and few macrophages. Other known materials, such as L-PLA and PGA, showed Macrophage as well as acute inflammatory cell reaction. The present invention provides the use of caprolactone monomers or poly-caprolactone and other lactone derivatives in certain configurations and applications to approximate an ideal solution for bioabsorbable fixation and interbody spinal devices. Low tissue reactivity and gradual absorbtion over a desired period, such as two years, provide the desired function of the present invention, as a bone column stabilizing implant.

More specifically, the material from which plate 12 and screw 16 are made are bioabsorbable materials that are non-toxic, moldable into various appliances, and absorb over an appropriate time interval while maintaining structural integrity sufficiently long to provide for adequate bone column healing. The epsilon caprolactone material has properties that are suitable as a bone stabilizer and a bone attachment implant. Specific data indicate low tissue reactivity, bioabsorbtion greater than 6 months, and lab testing (including saline bath at temperature) which shows excellent tolerance to load cycling through a variety of displacements and loads.

It is also contemplated in the invention to utilize a blend of polymers having a predetermined biodegradation rate. Such polymers may include those made utilizing the monomers of lactide, glycolide, co-polymers of these materials including L-lactide, D, L-lactide, glycolide, including: Poly(L-lactide-co-D,L-lactide), Poly(L-lactide-co-glycolide), and Poly(D,L-lactide-co-glycolide) para-dioxanone, trimethylene carbonate. Particularly, it has been found that such materials are useful for the screws 16. The polymers may be used to make copolymers having random, blocked or segmented block sequences, or combinations thereof.

As an example, it has been found that the device 12 can be formed of a molded e-caprolactone material, with plate 12 formed with dimensions to exceed FDA 510(b) standards, under a variety of appropriate loads and displacements including saline bath environment at body temperature. As a particular example of the materials used to provide the desired stability in connection with resorption, the plate 12 may be formed entirely of an e-caprolactone material, which is preferably predominantly crystalline. The monomer may have a melting temperature of between 50-70 degrees C., with an inherent viscosity of between 0.8-1.8, and more particularly between 1.1 to 1.7. The tensile strength is desired to be in the range of 1000-4000 psi, and more particularly around 3000 psi. The material has a shear strength in the range of 500-2500 psi, and more particularly around 1820 psi. The elongation at fracture may be in the range of 25% -500%, and more particularly around 42%. The modulus of elasticity of the material may be in the range of 20,000-100,000 psi, and more particularly around 58,000 psi. With the desired material, the resorption time is desired to be at least four months, but more particularly between 18-36 months, or around 24 months. The resorption rate desirably allows a predetermined degree of bone healing, such that upon initial resorption, the loads carried by the plate 12 are slowly transitioned to the surrounding bone during resorption.

With respect to the screws 16, the material is again desired to be resorbable, but desirably provides structural integrity to maintain proper placement of the plate 12 over an extended period of bone healing, and to facilitate proper connection to the bone segments. In a particular example, the screws 16 are constructed of a co-polymer of lactide monomers. The material may be a blend of L-lactide and D,L-lactide, such as in a ratio in the range of 50-80:50-20. For example, an amorphous co-polymer of 75/25 poly[L-lactide-co-DL-lactide] has been found to provide the desired characteristics. Such a material may have a melting temperature of between 100-200 degrees C., and more particularly around 180 degrees C. The material may have an inherent viscosity of between 0.6-8.0, and more particularly around 3.0. The tensile strength may be in the range of 2000-8000 psi, or more particularly around 5825 psi. The elongation at fracture may be in the range of 3%-300%, and more particularly around 15%. The modulus of elasticity of the material may be in the range of 20,000-400,000 psi, and more particularly around 26,714 psi. With the desired material, the resorption time is desired to be at least three months, but more particularly between 3-24 months, or around 12 months. The resorption rate desirably allows a predetermined degree of bone healing, such that upon initial resorption, the loads carried by the screws 16 are slowly transitioned to the surrounding bone during resorption.

Plates 12 may be used in various procedures as described in the prior applications incorporated by reference. The plate 12 may have a plurality of holes 14, which allow for the tapered screw 16 to be engaged with the vertebrae as shown in FIG. 1.

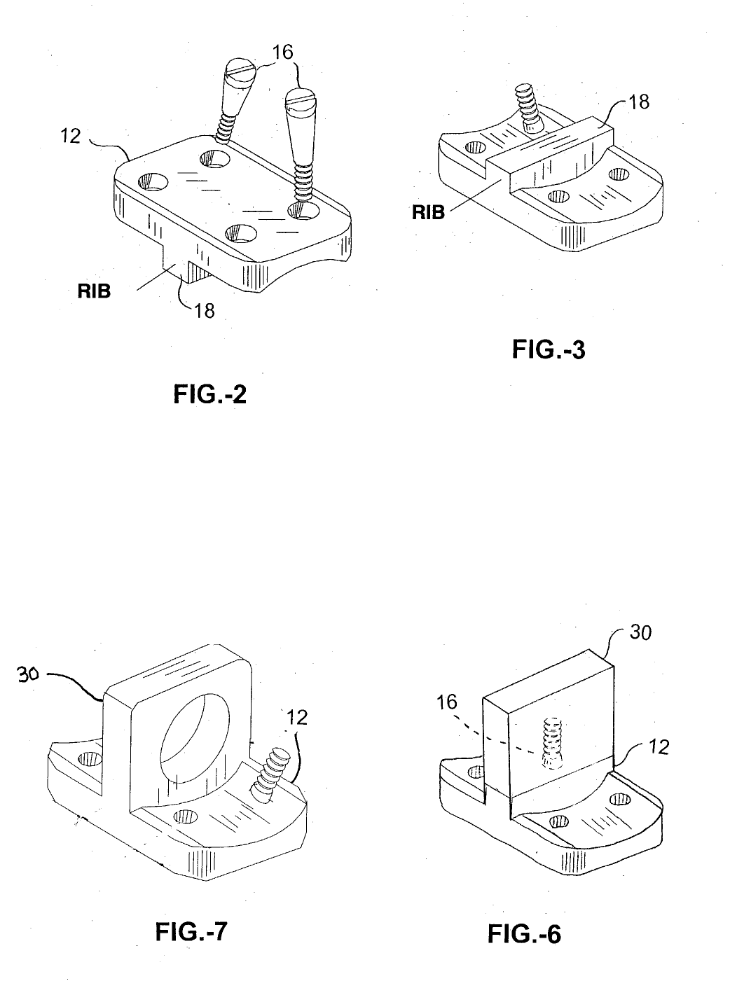

In FIGS. 2 and 3, an alternative embodiment of the plate 12 is shown, wherein the plate 12 is formed to include at least one reinforcing rib 18 associated therewith. As shown in these Figs., the plate 12 may be formed having a curvature on the posterior side, and a central rib 18 formed crosswise to the longitudinal axis of plate 12. In association with the configuration of plate 12, and the material from which plate 12 as well as screw 16 are constructed, there is formed a tight interference fit which reduces bending stress in the screw once associated with the plate. The screws 16 are also formed to have a tight interference fit in association with the vertebrae 20 and 22, which also reduces bending stress. Forming plate 12 of an e-caprolactone provides local resiliency in the area of the screw holes 14 to spread the load evenly between the plate 12 and screw 16 and allow for manufacturing tolerances. The invention provides a stabilization system wherein there is reduced wear on the screws 16 and holes 14, with less tendency for screws 16 to back out once inserted within the bone. Another unique aspect of the system 10 allows plate 12 to be formed into a customizable shape during a surgical process, by the application of heat and reforming pressure.

As seen in FIG. 4, a plate 12 and associated screw 16 are shown in-situ relative to a vertebra of an animal. A reinforcing rib 18 can be seen in the section. In use, the stabilization system 10 provided secure stabilization of the vertebral members, while providing essentially no reactivity with adjacent tissues. Further, essentially no inflammation was noted on pathology sections and Hematoxylin-Eosin staining. The fixation implant system 10 may be beneficial for patients with radiculopathy requiring bone column fusion. The device has been shown to be tolerant in vitro, and successful cervical fusion has been performed in test animals. The structural characteristics of the e-caprolactone material provide secure stabilization, while being resorbable so as to promote bone healing.

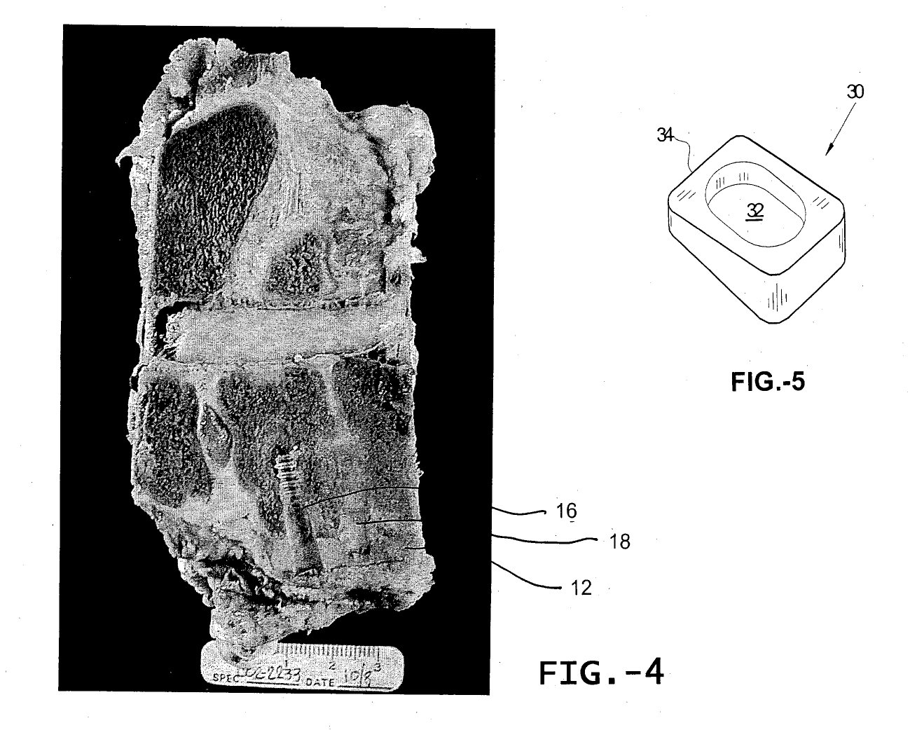

Turning to FIG. 5, a stabilization system comprising an interbody vertebral implant 30 is shown. The interbody implant 30 is designed for use as a cervical implant placed from an anterior approach. The interbody implant 30 will support bone segments in a bone column relative to one another in compression. The interbody implant 30 may be constructed of the e-caprolactone monomeric materials as described previously. The interbody 30 may also include one or more openings 32 in which a graft material may be positioned. The device or implant 30 allows for a graft material, such as a bone growth stimulating material, to be placed within the interbody implant portion 32. The graft may be a small piece of bone, a piece of calcium, a synthetic material, protein/DNS/gene strand or other material, to act as a bone growth enhancer. The interbody 30 is generally formed as a rectanguloid column, which has dimensions designed to provide compression support between bone segments of a bone column. The interbody implant 30 has a posterior side 34 to be positioned toward the posterior portion of a bone column, with an axis through the faces of the interbody implant 30 which substantially passes thru the central portion of adjacent bone segments. The interbody implant 30 may be secured in place by ligamentotaxis, screws, bonding agents or other bone attachment means. In an alternative embodiment as shown in FIG. 6, the interbody implant 30 is selectively attachable to a fixation plate 12, such as in accordance with the previous embodiments of the stabilization plate described herein. In this embodiment, the interbody implant 30 is selectively coupled to the plate 12 by means of a bioabsorbable screw 16. As a further alternative embodiment, the plate 12 can be formed with an integral interbody implant, such as shown in FIG. 7.

As seen in FIG. 8, the combination of a fixation plate 12 in combination with an interbody implant 30 provides a stabilization system that allows proper spacing and maintenance of the distance, geometry and continuity between bone segments 20 and 22. The plate 12 may be curved to conform to the vertebrae, and along the spinal axis to maintain lordosis. As seen in this Fig., the plate is curved to maintain lordosis. The interbody implant 30 may be integral or a separate member selectively attached to plate 12, and these members may be combined before or after implant placement. The interbody implant 30 can again provide a structural barrier to contain or limit tissue growth, or movement to protect or separate tissues and implants. Additional means to secure the interbody implant in a desired position may be provided, such as previously described. Additionally, ribs may be provided on the implant surfaces to provide increased rigidity to the implant. An opening may also be provided within implant 30 for the insertion of a bone growth promoting material or other graft material. The dimensions of the plate 12 and/or interbody implant 30 can be varied for a particular application, and due to the nature of the material, reshaping or reducing dimensions of these elements may be possible during a surgical procedure. As the material is a polymeric material, it is relatively easily manipulated in this manner.

In use, the combination of an interbody implant 30 in conjunction with a stabilization plate 12 provides a method for stabilizing a bone column by fixing of bone segments, with respect to one or more other bone segments, or with respect to another implant or graft material positioned within the bone column. As an example, the system may be applied by machining one or more holes in the bone column, and positioning an interbody implant between bone segments to a required depth. The implant is positioned to maintain desired bone column lordosis. Thereafter, a fixation plate may be attached to the implant, and secured to the adjacent bone segments to maintain the rotation and position of the implant. In this operation, it may be useful to temporarily secure the bone column in a desired position by means of metal screws or other fixation means. If necessary, the interbody implant and/or fixation plate is machinable or modifiable at the time of implantation, to conform to the bone column and/or the bone segment geometry. In this procedure, the implant may also be affixed to the bone segments or other tissue to temporarily or permanently serve as an anchor therefore. The interbody implant 30 according to the invention, either alone or in combination with affixation plate 12, may also serve as a bone cement restrictor for use in a variety of applications.

Although the present invention has been described with reference to the various embodiments thereof, various modifications and adaptations are considered to be within the scope of the invention and are contemplated thereby. The scope and protection afforded under the patent is therefore only limited according to the appended claims.

What is claimed is:

1. A biodegradable bone stabilization system comprising a stabilization member which is selectively positioned and secured relative to at least two bone segments within a bone column, to support one of the bone segments with respect to at least one other bone segment, implant or graft material within the bone column, for the purpose of maintaining distance, geometry and continuity between at least two bone segments, wherein the at least one stabilization member is formed of a material that provides structural support of at least a predetermined amount while concomitantly having a biodegradation rate such that the structural integrity of the implant is maintained for sufficient time to allow bone healing between the at least two bone segments, with subsequent complete removal from the bone column.

2. The bone stabilization system according to claim 1, wherein the bone stabilization member is a fixation plate adapted to be attached to adjacent bone segments.

3. The bone stabilization system according to claim 2, wherein the plate is attached to a bone segment by means of at least one fixation screw, pin or other attachment device.

4. The stabilization system according to claim 3, wherein a plurality of fixation screws attach the plate to adjacent bone segments, the fixation screws having a locking taper extending into the underlying bone for at least a predetermined distance.

5. The stabilization system according to claim 2, wherein the plate includes a longitudinal axis, and at least one rib member extending in an opposing direction to the longitudinal axis.

6. The bone stabilization system according to claim 1, wherein the bone stabilization member is an interbody implant adapted to be positioned between bone segments.

7. The stabilization system according to claim 6, wherein the interbody implant is formed as a rectanguloid body having dimensions to maintain the bone segment contact between adjacent bone segments.

8. The stabilization system according to claim 6, wherein the interbody implant includes at least one opening in which a graft material can be selectively positioned.

9. The bone stabilization system according to claim 1, wherein the at least one stabilization member is formed of an e-caprolactone monomer.

10. The bone stabilization system according to claim 9, wherein the e-caprolactone monomer has a tensile strength in the range of 1000-4000 psi, and a shear strength in the range of 500-2500 psi.

11. The stabilization system according to claim 1, wherein a first stabilizing member comprising at least one interbody implant is positioned between at least two bone segments, and a second fixation plate member associated with the interbody implant is selectively secured to at least one bone segment for positioning of the bone segment and interbody implant relative thereto.

12. The stabilization system according to claim 1, wherein the at least one bone stabilizing member provides a structural barrier to contain or limit tissue growth or movement, and to protect or separate tissues within a body.

13. The stabilization system according to claim 1, wherein the at least one bone stabilizing member is modifiable, substantially at the time of implantation to conform for the bone column and/or bone segment geometry.

14. A biodegradable interbody implant comprising,
a body member being configured to be positioned between at least two bone segments within a bone column, to support one of the bone segments with respect to at least one other bone segment, implant or graft material within the bone column, for the purpose of maintaining distance, geometry and continuity between at least two bone segments, wherein the interbody implant is formed of an e-caprolactone monomer material, the material having a biodegradation rate to provide sufficient structural integrity for an amount of time for bone healing between the at least two bone segments.

15. A biodegradable bone fixation plate implant comprising,
a plate member being configured to be positioned laterally adjacent and attached to at least two bone segments within a bone column, to fix the bone segments with respect to one another, wherein the fixation plate is formed of an e-caprolactone monomer, the plate having a biodegradation rate to provide sufficient structural integrity for an amount of time for bone healing between the at least two bone segments.

16. The fixation plate implant according to claim 15, further comprising a plurality of screw holes, wherein the plate is attached to the bone segments by a plurality of screws, wherein the screws are formed of a poly[L-lactide-co-DL-lactide] material.

17. The fixation plate implant according to claim 16, wherein the screw material is a blend of L-lactide and D,L-lactide, in a ratio in the range of 50-80:50-20.

18. The fixation plate implant according to claim 16, wherein the screw material has a biodegradation rate in the range of 3 to 24 months.

19. The fixation plate implant according to claim 15, wherein the plate has at least one reinforcing rib.

20. A method for stabilizing a bone column utilizing at least one biodegradable implant, the comprising the steps of,
positioning the implant relative to the bone column,
attaching the implant to position at least two bone segments in a desired position relative to one another, wherein the implant is formed of an e-caprolactone monomer material, the material having a biodegradation rate to provide sufficient structural integrity for an amount of time for bone healing between the at least two bone segments.
